(19) United States
(12) Patent Application Publication
(10) Pub. No.: US 2009/0203119 A1
(43) Pub. Date: Aug. 13, 2009

(54) BIOMASS REACTOR

(76) Inventor: Brian Anthony Evans, Johannesburg (ZA)

Correspondence Address: CHARTER IP, LLC, P.O. BOX 64, The Plains, VA, 20198, US

(21) Appl. No.: 12/375,040

(22) PCT Filed: Jul. 25, 2007
(86) PCT No.: PCT/IB07/52963

§ 371 Date: Jan. 26, 2009

(30) Foreign Application Priority Data
Jul. 25, 2006 (ZA) 2006/00711

(51) Int. Cl. C12M 1/00; H05B 6/02

(52) U.S. Cl. 435/298.1; 219/618

(57) ABSTRACT

A reactor for the conversion of biomass material includes an elongate annular chamber with an inner wall and an outer casing. A screw conveyor extends from one end into the chamber to feed biomass material. A heat source is provided within the inner wall of the chamber, and may be comprised of a series of permanent magnet assemblies mounted for rotation within a sleeve of electrically conductive material. The sleeve also serves as the inner wall of the chamber. Each magnet assembly includes a series of magnets spaced apart on a periphery of a circular support. A layer of insulating material is provided between the magnet assemblies and the sleeve. A rotatable shaft adapted for connection to a driving power source provides a mounting for the magnet assemblies.

### BACKGROUND

1. Field of the Invention

The example embodiments in general relate to a reactor for conversion of a biodegradable material through heating, and to means for generating heat in and from such a reactor.

2. Related Art

Large quantities of biodegradable waste material are generated from different sources and usually require to be disposed of. Such material is often buried or burnt without much benefit being obtained and often at high cost.

Particularly in rural areas where “clean power” is not available in the form of electricity, the environment is frequently spoiled by sewage, refuse and unwanted vegetation.

### SUMMARY

An example embodiment of the present invention is directed to a reactor for the conversion of biomass material. The reactor includes an elongate annular chamber having an inner wall and outer casing, a feed conveyor at one end of the chamber, an outlet at the other end, and a heat source located within the inner wall of the chamber.

In this example a heat exchanger is located around the chamber; and the conveyor is a screw conveyor extending partway along the chamber.

The heat source includes a permanent magnet assembly mounted for rotation within a sleeve of electrically conductive material. The sleeve may serve as the inner wall of the chamber. Additionally, a layer of insulating material may be provided between the magnet assembly and the sleeve. The chamber may have a non-magnetic outer casing.

There is provided a mounting for the magnet assembly. The mounting is a rotatable shaft adapted for connection to a driving power source. The magnet assembly includes a series of magnets spaced apart on a periphery of a circular support. In an example, a plurality of the magnet assemblies can be spaced apart along the shaft.

The example embodiment also provides for the sleeve to be formed from stainless steel; and for the casing to be formed from aluminum.

In an example, a computer connected to a thermocouple inside the chamber to control operation of the reactor. This includes controlling an air inlet valve in the chamber, rotation of the magnet assemblies and/or rotation of the screw conveyor.

Another example embodiment of the present invention is directed to a heat source for a biomass reactor. The heat source includes a permanent magnet assembly and a sleeve of electrically conductive material. The magnet assembly is mounted for rotation within the sleeve.

### BRIEF DESCRIPTION OF THE DRAWINGS

These and other features of the example embodiments will be appreciated from the following description, by way of example only, with reference to the accompanying drawings.

FIG. 1 shows a part cross-sectional side view of a biomass reactor.

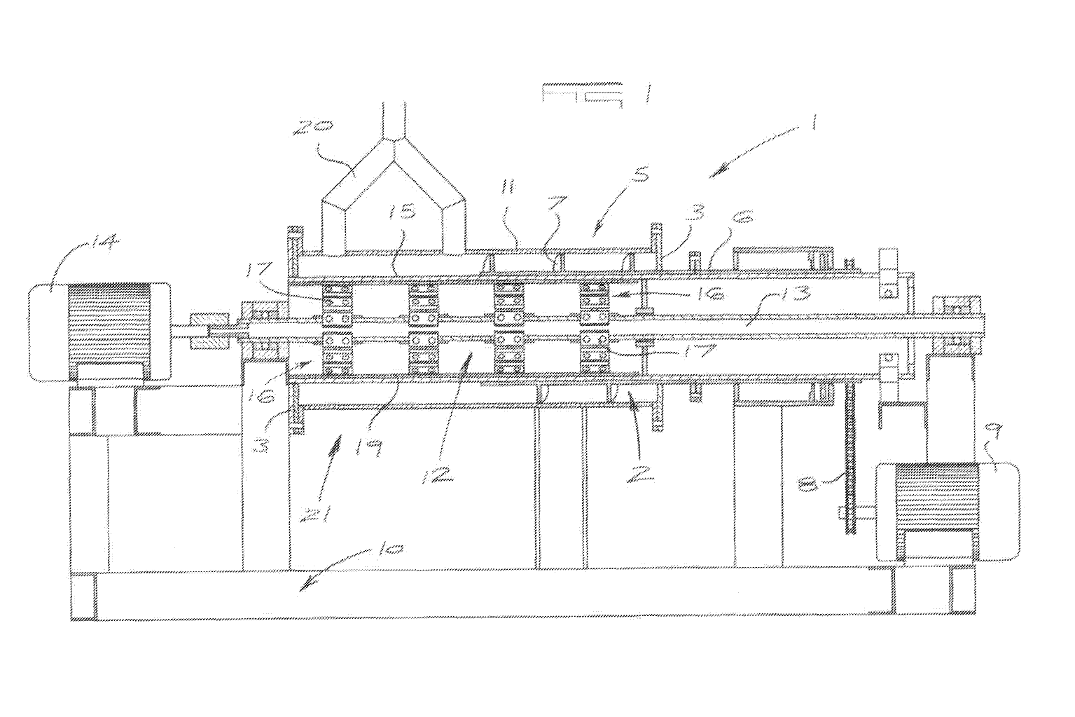

FIG. 2 shows a cross-sectional end view of part of the reactor.

FIG. 3 shows a perspective view of the reactor.

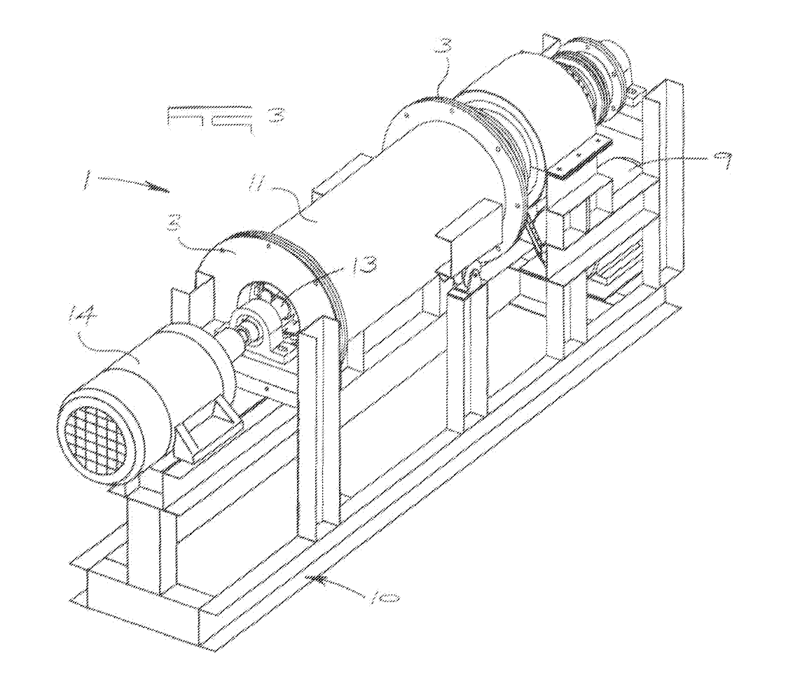

### DETAILED DESCRIPTION

Referring to the drawings, a biomass reactor (1) consists of a feed screw conveyor (2) which is located at one end of, and extending into, an annular chamber (4). An inlet at (5) adjacent the same end of the chamber (4) will feed into the screw conveyor (2).

The conveyor (2) includes a cylinder (6) with an outwardly projecting screw formation (7). This cylinder (6) is connected through a chain (8) to be driven by a first electric motor (9). The motor (9) may be of variable speed.

The reactor (1) is mounted on a suitable stand (10). The chamber (4) includes an outer casing (11) which contains the biomass material.

Reactor heating means (12) is provided within the core of the annular chamber (4). Also supported from the stand (10) and external to the chamber (4) is a shaft (13) mounted in bearings. The shaft (13) is connected to be driven by a second electric motor (14). The shaft (13) extends axially through an electrically conductive sleeve (15), which provides the inner wall of the chamber (4). Fixed for rotation with the shaft (13) are four spaced apart magnet assemblies (16). The ends of the reaction chamber (4) are closed with plates (3).

Each magnet assembly (16) is provided with permanent magnets (17) mounted from the shaft (13) to be rotated in close proximity of the inner surface of the sleeve (15) to provide the source (12) of heat for the chamber (4). The magnets (17) will preferably be of the kind known as rare earth, Neodymium-Iron-Boron (NdFeB) magnets. The sleeve (15) will be made of a stainless steel of suitable composition and the casing (11) will be made of non-magnetic material such as aluminum.

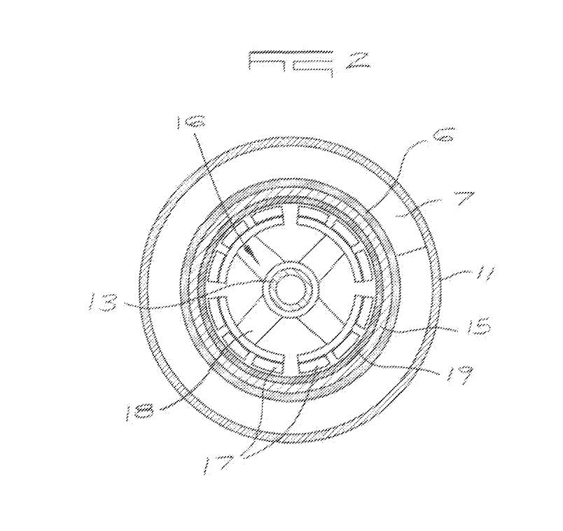

Each assembly (16) includes a circular support (18) with the magnets (17) carried on its periphery. This can be seen more clearly in FIG. 2. A layer (19) of insulating material is provided between the magnets (17) and the sleeve (15). The material can be of any suitable kind. It will provide the temperature difference required to prevent loss of magnetism due to heat exposure while allowing for desired heating at the sleeve (15). In particular, in this embodiment, it will allow the magnets (17) to be kept at a temperature below about 80° C. The sleeve (15) is open at its ends for ventilation of the magnet assemblies (16).

A flue (20) extends upwardly from the chamber (4) through which the producer gas will escape. A solid carbon outlet at (21) is provided in the floor at the end of the chamber (4) opposite the feed and a holding bin (not shown) will be provided for collecting the carbon.

In use, raw biomass is compressed and fed into the reactor (1) via conveyor (2) at a rate determined by its rotation. The speed of the conveyor (2) will be such that the compression of the biomass as it is moved along the reactor (1) forms an air seal to prevent the ingress of oxygen into the chamber where biomass pyrolysis will occur.

The shaft (13) is driven to rotate the permanent magnets (17) around in the sleeve (15). The magnet flux generates a short-circuited electrical current in the sleeve (15) which results in heating. This will heat the sleeve (15) to about 360° C. The heat generated is shielded from the magnets (16) through the layer (19) which will ensure that the magnets (16) are kept within a desirable temperature range preferably having a maximum temperature of about 60° C. The heat is transferred to the biomass at the outer surface of the sleeve (15).

As the biomass is driven along the chamber (4) by the conveyor (2), the temperature of the biomass reaches exothermic temperature. An external fuel source may be used to start the reaction such as liquid petroleum gas. This becomes redundant once exothermal temperature is reached. Carbon is delivered to the holding bin through discharge outlet and exits the system for stabilization. The producer gas generated in the chamber (4) will be flared.

The continuous reactor (1) operates at a pressure slightly above ambient. This deters against the introduction of oxygen at the carbon discharge point.

A first thermocouple in the chamber (4) is connected to an onboard computer. The computer controls speed of rotation of the magnets (17) to maintain the exothermic temperature. This would be from about 350° C., which is where the exothermic reaction of biomass contents starts, up to about 400° C. The exothermic reaction maintains the required heat with losses to insulation made up by the magnet assemblies (16).

A fan (not shown) will be located at one end of the chamber (4) in line with the magnet assemblies (16). A second thermocouple in the magnet chamber switches on the fan when the temperature exceeds 60° C. The magnets (17) referred to demagnetize at about 90° C. The fan is driven by a back-up battery in the event of a power outage. This will save the magnets (17) from the heat that would otherwise migrate.

As a development to the embodiment thus far described, in place of insulation which would usually surround the reaction chamber (5) there is a heat exchanger (not shown). The heat exchanger is provided as a jacket around the chamber through which a suitable fluid, heat transfer medium can be circulated. The medium may be water, a mixture of water and something else, or any other suitable liquid. The thermal energy of the reaction is transmitted to the heat transfer medium via the heat exchanger. This energy can then be used for any of a number of applications requiring heat.

The heat exchanger will be configured to provide a suitable heat transfer surface area and is of such a construction to facilitate heat exchange that is as effective as possible. The energy in the biomass reactor (1) thus serves as a heat source. The magnets (17) alone would not be able to sustain the level of heat without the exothermic reaction.

The exothermic reaction temperature is, as already mentioned, at about 350° C. and carbon is produced by the reactor under these circumstances. Where carbon is not required as a product, a small amount of air can be admitted to the chamber (4). Apart from this, the chamber (4) would otherwise be substantially oxygen free. The solid carbon combusts spontaneously in this environment. The rate of burn is proportional to the amount of oxygen allowed into the chamber. The reaction temperature will rise from 400° C. to 600° C. For power generation, a better temperature differential is required between the chamber (4) and the heat exchanger. Here, instead, all the energy is dissipated through full combustion at 600° C. (This could be taken to about 1200° C. but associated problems with the component materials become a risk.)

The biomass is reacted completely to generate heat rather than to recover carbon or gases. Ash is predominantly discharged at the outlet (21). The thermal energy of the flared gases can also be used to heat the transfer medium. Suitable components for the recovery of this energy will be within the design competence of a suitably skilled person.

The oxygen enters the chamber (4) through a valve (not shown) which is controlled by the computer. The computer, which is connected to the first thermocouple, controls the chamber (4) temperature in this manner. The computer will also control the speed at which the conveyor (2) rotates. The rate of feed of the biomass may thus also be varied by the computer to maintain a required temperature.

With the developments in permanent magnets, there are some kinds which only demagnetize at about 120° C. Such magnets would be better suited to the latter described application.

The plant above described is uncomplicated and simple to use and maintain. A suitably skilled person will appreciate that a number of variations may be made to the features of the described embodiment without departing from the scope of the example embodiments of the current invention.

1. A reactor for the conversion of biomass material comprising:
an elongate annular chamber having an inner wall and an outer casing,
a feed conveyor at one end of the chamber,
an outlet at the other end of the chamber, and
a heat source located within the inner wall of the chamber.

2. The reactor of claim 1, further comprising a heat exchanger located around the chamber.

3. The reactor of claim 1, wherein the conveyor is a screw conveyor extending partway along the chamber.

4. The reactor of claim 1, wherein the heat source includes a permanent magnet assembly mounted for rotation within a sleeve of electrically conductive material.

5. The reactor of claim 4, wherein the sleeve serves as the inner wall of the chamber.

6. The reactor of claim 4, further comprising a layer of insulating material provided between the magnet assembly and the sleeve.

7. The reactor of claim 1, wherein the chamber includes a non-magnetic outer casing.

8. The reactor of claim 1, further comprising a mounting for the magnet assembly that includes a rotatable shaft adapted for connection to a driving power source.

9. The reactor of claim 8, wherein a plurality of magnet assemblies are spaced apart along the shaft, each magnet assembly including a series of magnets spaced apart on a periphery of a circular support.

10. The reactor of claim 5, wherein the inner wall is formed from stainless steel and the casing is formed from aluminum.

11. The reactor of claim 1, wherein a computer connected to a thermocouple inside the chamber controls operation of the reactor.

12. A heat source for a biomass reactor, comprising:
a permanent magnet assembly, and
a sleeve of electrically conductive material the magnet assembly mounted for rotation within the sleeve.

13. The heat source of claim 12, wherein the sleeve serves as an inner wall of a reactor chamber.

14. The heat source of claim 12, further comprising a layer of insulating material provided between the magnet assembly and the sleeve.

15. The heat source of claims of claim 12, further comprising a mounting for the magnet assembly that includes a rotatable shaft adapted for connection to a driving power source.

16. The heat source of claim 15, wherein a plurality of magnet assemblies are spaced apart along the shaft, each magnet assembly including a series of magnets spaced apart on a periphery of a circular support.
